(19) United States
(12) Patent Application Publication
(10) Pub. No.: US 2015/0182381 A1
(43) Pub. Date: Jul. 2, 2015

(54) SOLAR POWERED EVAPORATIVE COOLED WELDING HOOD

(71) Applicant: PAUL DIXON DAVIS, CARTHAGE, TX (US)

(72) Inventor: PAUL DIXON DAVIS, CARTHAGE, TX (US)

(21) Appl. No.: 14/307,084

(22) Filed: Jun. 17, 2014

Related U.S. Application Data
(60) Provisional application No. 61/964,302, filed on Jan. 2, 2014.

(51) Int. Cl. A61F 9/06; A42B 3/28; A42B 3/04

(52) U.S. Cl. CPC A61F 9/068; A42B 3/0406; A42B 3/286

(57) ABSTRACT

A welding hood with a solar powered evaporative cooling system that has a magnified solar panel produced by the light from the welding arc passing through a clear lens, the solar panel, and magnification lens; then bouncing off the reflective backing and back through the solar panel increasing the power of the solar panel and extending the life of the batteries that by an on/off switch operate a fan with an attached filter. This provides filtered circulating air that flows across the head of the welder wearing a damp cap liner or other preferred product; and that produces evaporative cooled air that cools the welder's head, clears the line of sight; provides an improved breathing area for the welder, and reduces related heat injuries and respiratory problems.

### CROSS-REFERENCE

This application is related to U.S. Provisional patent application number 61/964,302 filed on Jan. 2, 2014.

### STATEMENT REGARDING FEDERALLY SPONSORED RESEARCH OR DEVELOPMENT

“Not Applicable”

### REFERENCE TO SEQUENCE LISTING, A TABLE, OR A COMPUTER PROGRAM LISTING

### Compact Disc Appendix

“Not Applicable”

### BACKGROUND OF THE INVENTION

The present invention relates to welding hoods. Specifically, the present invention pertains to a solar powered evaporative cooled welding hood. Some welding hoods have a less desirable way of combining components than the present invention. Welders have worked in locations with little working space, such as bell holes, where the temperature can be extremely hot and where air flow is almost nil. Welders are vulnerable to heat stroke, heat rash, heat exhaustion and heat cramps. Also, welders are vulnerable to respiratory diseases caused by noxious fumes. Perspiration and dust particles interfere with the welder's sight.

Therefore, components capable of working together that cool the interior of a welding hood, and that reduce heat related injuries and respiratory diseases would benefit the welder. Thus, there still remains a considerable need for a solar powered evaporative cooled welding hood for the comfort and safety of the welder.

Cited references are U.S. Pat. No. 5,896,579 Johnson Apr. 27, 1999; U.S. Pat. No. 7,534,005 Buckman May 19,2009; U.S. Pat. No. 5,029,342 Stein Oct. 6, 1981 and U.S. Pat. No. 4,890,335 Crowson Jan. 2,1990.

### BRIEF SUMMARY OF THE INVENTION

In view of all the disadvantages a welder has been accustomed to working under, the present invention provides a welding hood with systems for creating solar powered evaporative cooled air.

Therefore, it is the object of the present invention to provide a welding hood that reduces the disadvantages of welding and that receives the advantages of solar powered evaporative cooled air for longer periods of time, while cooling a larger portion of the body and maintaining a safe environment by combining components in a unique way.

The components are: a welding hood, a pivotally adjustable head band, a clear lens, a magnification lens, a solar panel, a reflective backing, rechargeable batteries, a fan, a switch, an attachable filter and a damp cap liner or other preferable product.

The welding hood covers the front of the face, both sides of the face and the top of the welder's head. The pivotally adjustable head band is mounted to the welding hood. The dear lens, the magnification lens, the solar panel and the reflective backing together form a layered unit; wherein the dear lens is the front layer, the magnification lens is the second layer, the solar panel is the third layer and the reflective backing is the back layer. As a layered unit it is inserted into a casing mounted into the front of the welding hood. A light from the welding arc passes through the first three layers, becomes magnified, bounces off the reflective backing, which reflects the light back through the solar panel, thus increasing the power of the solar panel; wherein charging and extending the life of the rechargeable batteries. The filter is attached over the fan which is mounted into the top of the welding hood. The fan blows filtered air across a damp cap liner or other preferable product worn by the welder, cools the welder and blows the filtered air downward, reduces noxious fumes and dust particles for improved breathing and clearer vision. Thus, providing a solar powered evaporative cooled welding hood.

### BRIEF DESCRIPTION OF DRAWINGS

FIG. 1 shows a side view of an exemplary embodiment of the present invention.

FIG. 2 shows the hood's internal cavity of the exemplary embodiment, wherein the clear lens, solar panel lens, rechargeable batteries, switch, and a fan of the present invention are shown.

FIG. 3 shows a view of a cross section of the solar panel with its four components; the clear lens, magnification lens, solar panel, and the reflective backing.

FIG. 4 shows a view of a cap liner, though other preferable products could be used such as a handkerchief or bandana.

FIG. 5 shows a view of the filter and fan.

### DESCRIPTION OF THE PREFERRED EMBODIMENTS

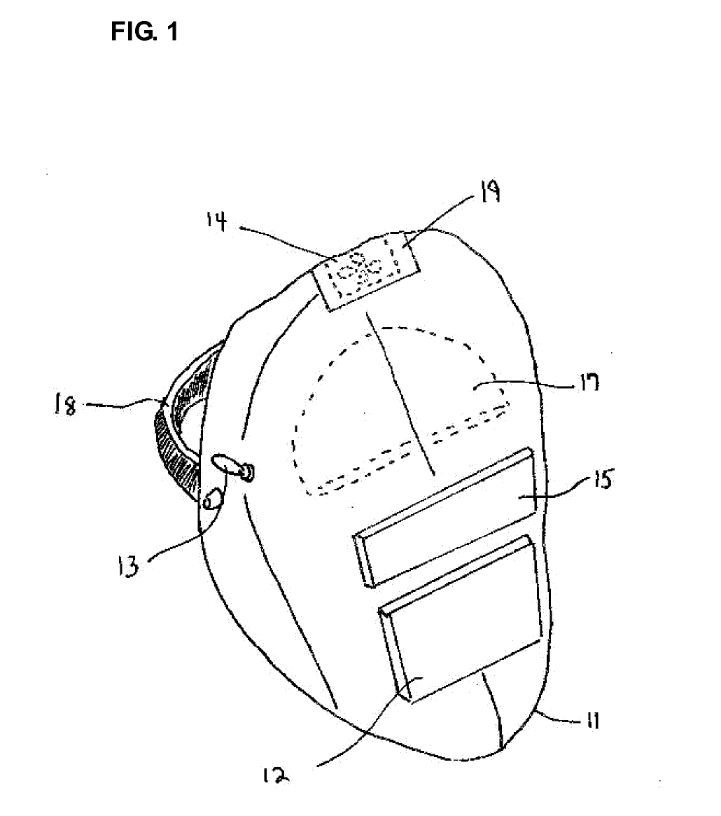

Referring now to FIG. 1, there is shown a side view of an exemplary embodiment of the solar powered evaporative cooled welding hood of the present invention. The present invention provides a welding hood 11 that is ideal for use in high temperatures and working conditions, wherein the temperature could cause the user to become vulnerable to heat stroke, heat rash, heat exhaustion and heat cramps. The present invention provides a welding hood 11 with continuous forced air means 14, with a filter 19 and a cooling means 17. This provides filtered circulating air, which flows across a cooling means 17, producing evaporative cooled air that cools the user's head. This also clears the line of sight, provides a clear breathing area underneath the welding hood 11, reduces heat stroke, other related heat injuries and respiratory problems. Further providing an interior hood environment that is pleasant and cooling to the user, which is particularly welcomed when in use in extreme heat. In an exemplary embodiment of the present invention, the welding hood 11 includes a built-in air flow 14 and quick easy access on/off switch 13 that does not restrict, limit or change the hood's primary function of providing protection for the user and an eye shield 15 against the light of the welding operation. The first goal is protection, followed by improvement of the hood's interior environment in hot air, while air is finally circulated to cool the user and remove noxious fumes from the hood's interior as a result of the welding process.

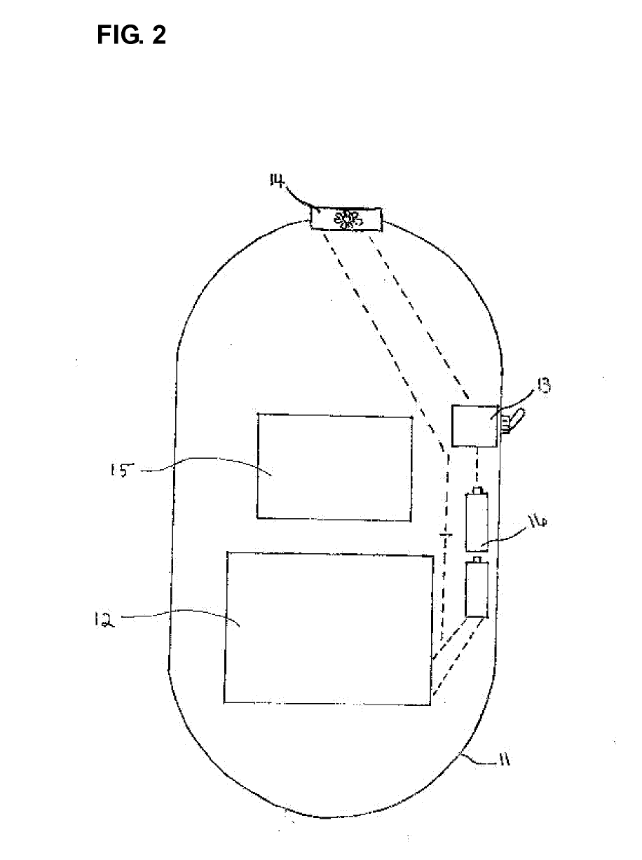

FIG. 2, is an interior view of an exemplary embodiment of the solar powered evaporative cooled welding hood of the present invention. The present invention provides a welding hood 11 housing the magnified reflective backing solar panel 12 that charges rechargeable batteries 16.

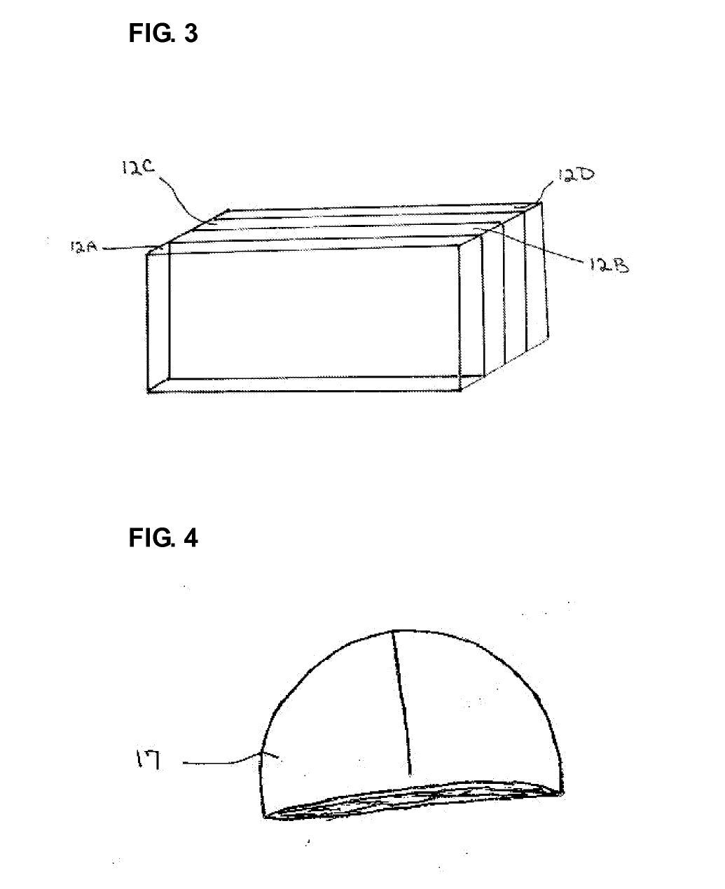

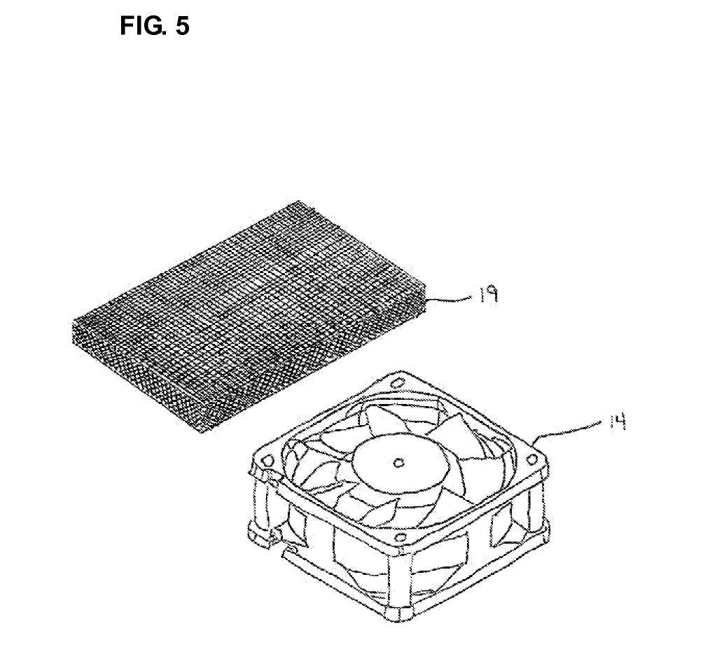

The rechargeable batteries 16 are used to give power to a fan 14 which can be turned on and off by a switch 13. The magnified reflective backing solar panel 12 can use sunlight, a 60 watt light bulb or a welding arc to charge the rechargeable batteries 16. FIG. 3, shows a cross section view of the magnified reflective backing solar panel. There are four layers; the clear lens 12a, the magnified lens 12b, the solar panel 12c, and the reflective backing 12d. The clear lens 12a is for protection of the assembly. The solar panel 12b draws power from sunlight, a 60 watt light bulb or a welding arc to charge the rechargeable batteries. The magnification lens 12c is to magnify the power that the solar panel 12b draws. The reflective backing 12d is to amplify the entire process of the solar panel 12b and magnification lens 12c.

The applicant certifies that the substitute specification contains no new matter.

What is claimed:

1. A solar powered evaporative cooled welding hood comprising: a welding hood having a pivotally adjustable head band being mounted to the hood; a viewing lens protecting a welder's eyes from a light from a welding arc; a solar powered system; an evaporative cooling system; an interior surface and an exterior surface; wherein the exterior surface encloses the front, right side, left side and top of a welder's head.

2. The solar powered system of claim 1 comprising: a clear lens; a magnification lens; a solar panel and a reflective backing; wherein forming a layered unit; wherein the clear lens being the front layer; the magnification lens being the second layer; the solar panel being the third layer and the reflective backing being the back layer; wherein the layered unit being inserted into a casing mounted into the front of the hood.

3. The light from the welding arc of claim 1 passing through the clear lens; the magnification lens and the solar panel of the layered unit of claim 2, being magnified; bouncing off the reflective backing; wherein reflecting the light back through the solar panel; wherein the solar panel power is increased.

4. The evaporative cooling system of claim 1 comprising a plurality of rechargeable batteries being mounted on the interior surface of the welding hood; a fan being mounted over an opening on the top of the welding hood; a switch being mounted into the interior surface of the welding hood; a filter being attached to the fan; and a damp cap liner or other preferable product worn on the welder's head.

5. The solar panel power of claim 3 wherein extends the life of the rechargeable batteries;
wherein rotating the fan blades; wherein blowing filtered air across the damp cap liner or
other preferable product cooling the welder and blowing the filtered air downward; reducing
noxious fumes and dust particles for improved breathing and clearer vision.
